(19) United States
(12) Patent Application Publication
(10) Pub. No.: US 2021/0290810 A1
(43) Pub. Date: Sep. 23, 2021

(54) MULTI-STAGE PROCESS CHALLENGE DEVICE, INDICATOR SYSTEM AND PROCESS CHALLENGE DEVICE SYSTEM

(71) Applicant: GKE GmbH, Waldems (DE)

(72) Inventor: Ulrich KAISER, Glashütten (DE)

(73) Assignee: GKE GmbH, Waldems (DE)

(21) Appl. No.: 17/259,485

(22) PCT Filed: Aug. 21, 2018
(86) PCT No.: PCT/EP2018/072552

§ 371 Date: Jan. 11, 2021

(51) Int. Cl. A61L 2/28; C12Q 1/22

(52) U.S. Cl. CPC A61L 2/28; C12Q 1/22

(57) ABSTRACT

Process challenge device (2), especially for simulating the worst-case penetration conditions of a load inside a sterilization chamber, comprising a detector volume (90) for housing a biological, chemical or physical indicator (140), whereby said detector volume (90) is arranged at a dead end of a series of gas volumes (110, 14, 118), whereby said process challenge device (2) comprises two parts (6, 14), namely a housing part (6) and an insertion part (14), whereby said insertion part (14) is at least partially insertable into said housing part (6) in such a way that said detector volume (90) is arranged without any seal inside said both parts (6, 14).

### FIELD OF THE INVENTION

The invention refers to a process challenge device, especially simulating the worst-case penetration conditions of a load inside a sterilization chamber housing a biological, chemical, or physical indicator arranged in a detector volume which is arranged at the dead end of a series of volumes in which gas flow is transferred in both directions. It also refers to an indicator system and a process challenge de-vice combination.

An indicator system is defined according EN ISO 11140-1 as a combination of a process challenge device and a detector inside which could be a biological, chemical, or physical indicator as a detector to monitor the presence of the sterilant. It is used as a surrogate model to represent the worst-case penetration conditions in-side of a sterilization load including its packaging. Since biological or chemical indicators cannot be put into the worst-case penetration locations inside of complex instruments, it is used as a surrogate model to check if the sterilization conditions are achieved in the load, represented by the surrogate indicator system.

### BACKGROUND

For aseptic operations in hospitals and sterile filling in industry the use of sterile instruments or materials is absolutely necessary. At a therefore necessary sterilization the sterilizing agent, for example steam, formaldehyde, ethylene oxide, hydrogen peroxide and/or ozone, is usually transferred via the gas phase to the sur-face of the instrument to be sterilized, to assure the total kill of existing germs. For this purpose, sterilizers with sterilization chambers are normally used in which the instruments or their materials are—always packaged—before they are sterilized. Before sterilization, the sterilization chamber requires first air removal and then is flooded with gaseous sterilant—also named sterilizing agent—, which requires that air inside has to be removed before. The sterilization agent shall contact all sur-faces of the instruments or materials to be sterilized so that the total kill of all germs occurs.

As the complete sterilization of the goods at all surface areas is only guaranteed if the sterilization agent reaches all external and interior surfaces, for example porous packs or hollow devices, like tubes and minimal invasive instruments. The removal of the air inside the goods and inside the sterilization chamber has to be secured by a suitable air removal procedure before the sterilization process starts. After that the sterilization chamber is flooded with the sterilization agent in order to reach all surfaces of the instruments inside the sterilization chamber with the sterilization agent. This is only possible when the complete penetration of the sterilization agent is assured to all surfaces through the packaging and geometric design of the instruments.

The complex structure of minimal invasive surgical (MIS) instruments is known to be problematic in sterilization processes. More and more medical devices are used with relatively long pipes or tubes and comparatively small free cross sections, so that a reliable surface contact of the sterilization agent to all interior sur-faces is made more difficult if there are remaining inert gases, like air, present. Furthermore, materials and goods with complex interior surfaces, for example textile packages, require sterilization. In such cases existing accumulations of remaining air or other non-condensable gases (NCG) may prevent complete or part con-tact of those surfaces. The complete sterilization is only assured when the air in-side the goods is completely removed before the sterilization process, and/or, when during the vacuum stage no air enters through leaks and/or no NCG are introduced into the sterilization chamber with the sterilization agent, to assure that the sterilization agent can reach all surfaces.

As all instruments are packed and therefore sterility cannot be tested directly be-fore use, the validation of sterilization processes before start up and routine monitoring during the sterilization process are necessary. In addition, detectors are used to prove the success of the sterilization process. For example, chemical indicators are used which change their colour when the indicator has been exposed to all critical variables and their critical parameters of the sterilization process, for example with condensing steam and temperature over time. Alternatively, or additionally, biological indicators may be used in form of stripes, suspensions, or self-contained biological indicators (SCBI). After a sterilization process the inserted indicators are tested to release the load.

Such chemical or biological indicators monitor that all critical sterilization variables and parameters of the process have occurred at the place where the indicator in-side the sterilization chamber is positioned. Such indicators cannot be placed at those critical areas of inaccessible surfaces of complex instruments and is no direct proof of the sterilization success possible. Therefore, surrogate test devices are necessary and are sterilized together with the goods outside the packs, to determine the success of the sterilization. For example, for the sterilization process of textiles or other materials, from Bowie and Dick a standard test pack was de-scribed (Bowie, I. W., e.a., The Bowie+Dick autoclave tape test, Lancet, 1963, p. 585-587), in which a chemical indicator test sheet of DIN A4-size has been placed centrally in a cotton package of 7 kg weight according to EN 285. Though this standard test is not exactly reproducible because of the cotton quality, cotton his-tory and individuality of the packages, and its penetration characteristic is different from hollow devices.

Alternatively, so called indicator systems can be used. In such a test device system, as described for example in EP 0 628 814 Al or in EN 867-5, the difficult accessible inside surface of complex instruments is simulated by a suitable model, enabling to monitor the success of the penetration processes into complex instruments in an analogous way.

Those well-known indicator systems consisting of “process challenge devices” (PCDs) and of a suitable detector to prove the penetration of the sterilization agent, connected to a suitably chosen length of tube at the gas entering side, which is open at its admission end as described in EN ISO standard 11140-1. This indicator system simulates the penetration characteristics of similarly designed

instruments which are supposed to be sterilized, where especially during an alternate gas exchange according a fractionated vacuum and/or the condensation of steam eventually remaining air or other non-condensable gases at the tube end in the area of the detector are concentrated.

If the detector of such a system connected to the tube end detects sterilization agent, it can be assumed, that—using a PCD with higher penetration characteristics—the instruments most inaccessible points of their inner surfaces must have been in contact with sterilization agent as well. Such a tube model as a test device which can hold for example biological or chemical indicators as a detector is also intended for the verification of sterilization processes in Euro Standard EN 867-5. To check the sterilization success of more complex goods, test devices of a different construction which are in their dimension suitably adapted, can be used, as described for example in the Euro Standards EN 285, EN 14180, EN 1422 or EN 867-5. The devices need to be long enough to provide a valid simulation of instruments.

Test devices described above are for example known from EP 0 628 814 Al or from EP 1 172 117 A2. A multi-staged test device is known from the

EP 1 468 701 B1. In this test device, the gas-collection-volume is designed in a multi-stage way so that cross section/volume of each stage decrease between neighbouring volumes towards the direction of the detector volume.

The detector volume is positioned at one end of the test device channels while the entrance to the gas collection volume is arranged, for instance, at the other end of the channel of the test device. The detector volume can be opened such that the detector can be placed inside the detector volume and be closed again. When the test device is placed in the sterilisation chamber the detector volume is exposed to sterilisation agent. In order to prevent direct entry of sterilisation agent into the detector volume, which would nullify the test, a sealing element has to be provided to seal the region where the detector volume can be opened. The sealing element, which is for example built as a gasket, wears off over time and needs to be replaced.

An undetected malfunction leads to non-valid test results. This invention totally eliminates this risk.

An indicator system is defined according EN ISO 11140-1 as a combination of a process challenge device and a detector inside which can be a biological, chemical, or physical indicator as a detector to monitor the presence of the sterilant. It is used as a surrogate model to represent the worst-case penetration conditions in-side of a sterilization load including its packaging. Since biological or chemical indicators cannot be put into the worst-case penetration locations inside of complex instruments, it is used as a surrogate model to check if the sterilization conditions are achieved in the load, represented by the surrogate indicator system.

### SUMMARY OF THE INVENTION

It is an object of the invention to improve a test device in such a way that reliable test results are obtained and maintenance as well as spatial extensions of the de-vice are reduced. Additionally, an improved indicator system using an improved process challenge device is provided.

The object of the invention is achieved by a process challenge device, especially for simulating the worst-case penetration conditions of a load inside a sterilization chamber, comprising a detector volume for housing a biological, chemical or physical indicator, whereby the detector volume is arranged at a dead end of a series of gas volumes, whereby the process challenge device comprises two parts, namely a housing part and an insertion part, whereby the insertion part is at least partially insertable into the housing part in such a way that the detector volume is arranged without any seal inside both parts.

Preferred embodiments of the invention are specified in the dependent claims and the figure description.

The invention is based on the consideration that common disadvantages of known test devices are the need of sealing of the detector chamber and the needed space of a combined test device like a Bowie-Dick cotton test pack. For these known devices, a varying of the volume is not possible. Common test devices only achieve a high sensitivity with an adequately long-dimensioned gas collection volume at the cost of the compactness.

Applicant has found that these disadvantages can be removed by building process challenge device which comprises two parts of which one part provides an outer housing or casing and the other part is insertable into the other part. Arranging the detector volume at the last or final of these stages removes the need of a sealed PCD since it is only accessibly the sterilizing agent which has travelled all the way from the entrance region through all stages to the detector volume. By design, no alternative way of accessing the detector volume is possible. Such a design also provides the possibility to vary the difficulty of the total gas passage. That the detector volume is arranged inside both parts preferably means that it is in each spatial direction surrounded by both parts. Preferably, the detector volume is arranged in an end piece or duct of insertion part 14. The indicator/detector arranged in detector volume 90 can then be easily accessed by pulling out insertion part 14 from housing part 6.

The process challenge device preferably comprises two or more parts, among them an insertion part and a housing part, whereby the insertion part is insertable into the housing part, and whereby no sealing is provided between these two components.

The insertion part and the housing part themselves respectively can be built from one or more components which are connected to/fixed to each other. They are preferably built of metal, plastic, or a metal/plastic composite material.

An important feature of the process challenge device according to the invention is that it comprises no sealing elements/gaskets. In this way, the process challenge device has a long lifetime and high reliability since no risk of an untight/leaky sealing is present.

Preferably the two parts fit into each other without sealing and form, combined together, in series connected volumes for providing specific penetration characteristics for sterilization agents.

Advantageously, the series of volumes provides connected channels, whereby one end of these channels is always connected to a sterilization chamber and the other end is connected to the detector volume which is configured to house, alternatively one or more chemical, biological, or physical indicators for detecting the presence of a sterilizing agent.

Preferably the series of gas volumes is provided inside the respective parts and/or between the two parts. A gas volume is therefore limited by surfaces/walls of the insertion and/or housing part. It can be positioned fully inside one of the two parts or at least partially between surfaces/walls of both parts.

The two parts advantageously comprise means for a force-fit and/or form-fit connection, especially by clicking or snapping together.

In a preferred embodiment, adjusting means are provided for positioning the two parts with respect to each other in a plurality of defined positions with respect to each other.

Preferably, the length and/or volume of at least one gas volume increases when the insertion part is pulled out from the housing part, especially when it is pulled out and then adjusted in a defined position. In this way, the sensitivity of the process challenge device can be adjusted in a reproducible way among series of tests.

In a preferred embodiment, the detector volume is arranged at an end piece of the insertion part. When the insertion part is fully extracted from the housing part, the detector volume can conveniently be accessed for removing/inserting the indica-tor/detector.

The detector volume preferably is accessible only by gas which has travelled through the series of gas volumes to the dead end. This means that the detector volume has no other gas access which, as in other known device, needs a seal to prevent gas entry.

Advantageously, between the insertion part and the housing part an entrance region for sterilizing agent to enter the series of gas volumes is provided. The entrance region is preferably arranged between a cover part of the insertion or second part and the hull/casing which is provided by the housing or first part. This entrance region is especially advantageous for the embodiment with adjusting means for adjusting the relative longitudinal position between housing and insertion parts.

The housing and/or said insertion part are preferably made of metal, plastic, or metal-plastic-bonded system.

In a preferred embodiment, from an entrance region to the dead end the gas volumes decrease in volume and/or cross section.

In a preferred embodiment, the insertion part and/or housing part comprises a round, oval, rectangular, or multi-edge shape/cross section. In this way, the necessary vertical space of the test device can be optimized and reduced. In a preferred embodiment, the casing or housing part has a circular shape.

Preferably the series of gas volumes is at least partially filled with porous material. Especially preferably, the whole or essentially the whole series of gas volumes is filled with porous material.

The detector volume preferably has a volume of less than 200 microliters, a length between 3 and 6 cm and a cross section between 3 and 5 mm2. In this way, the detector volume is designed for providing an optimized sensitivity for the sterilizing properties of a sterilizing process.

The gas volumes advantageously are arranged spatially nested in a zig-zag-manner. The term zig-zag in generalized manner denotes a passage or canal or staged volume with a sequence of passageways which at least partially run parallel to each other. It especially denotes a nested back and forth of passageways.

Preferably, the spatially nested gas volumes comprise a series of predetermined passageways of which a subsequent passageway along the passage is located radially within the previous passageway, and whereby the detector volume is arranged at the end of the last passageway of this series of passageways. The passageways are therefore arranged subsequently nested within each other, which allows providing a compact design of the test device. In order for sterilizing agent to reach the detector volume, it has to pass through all stages or passageways.

Advantageously, the test device comprises a series of three passageways. These passageways are arranged in a zig-zag way inside the casing. The three passageways are aligned parallel to each other. In order to reach the detector volume, the sterilizing agent moves/flows in the first and third passageway in the same di-rection and in the second passageway in an opposite direction.

Preferably, the process challenge device comprising a series of three passage-ways. These passageways are provided by three gas volumes.

In a preferred embodiment, the test device comprises an outer casing with a first closed end and a second end comprising the entrance region, whereby radially inside the outer casing a first duct is arranged such that a first passageway is de-fined between the first duct and the outer casing from the entrance region to the first end.

Preferably radially within the first duct a second duct is arranged defining a second passageway, whereby a first inner entrance region is defined at the first end and whereby a second passageway is defined from the first inner entrance region be-tween the first and second ducts towards the second end.

Preferably radially within the second duct a third duct is arranged, whereby a second inner entrance region is defined between the second duct and the third duct at the second end, and whereby a third passageway is defined from the second end to the first end between the second duct and the third duct, and whereby at the end of the passageway at the first end, the detector volume is arranged.

Preferably the diameter decreases between ducts of subsequent stages.

Preferably the cross section decreases between subsequent passageways. The cross section denotes the free cross section, i.e. the cross section which is available for the fluid to flow. Towards the detector volume, the cross section between subsequent passageways preferably decreases at least by 50%, more preferably by more than 75%.

Preferably the volume decreases between subsequent passageways. The ducts are preferably arranged concentric to a common middle axis.

The test device preferably comprises entrance means for adjusting the size of said entrance region. The test device preferably comprises varying means for adjusting the length of at least one passageway.

Preferably varying means, especially snap means and/or notches, are provided by which at least one passageway can be set to a plurality of defined lengths.

In a preferred embodiment, the test device comprising two parts, especially as described above, of which a second (insertion) part is insertable into the other (housing) part, whereby the varying means allow varying the degree by which the insertable part is inserted into the housing part.

The object of the invention is also achieved by an indicator system, comprising a process challenge device described above and at least one chemical, biological, or physical indicator/detector arranged in the detector volume.

Preferably the indicator system comprises a biological indicator, whereby a carrier of the biological indicator is made from paper, metal, glass fiber, plastic, stainless steel, any plastic foil, Tyvek or any combination, especially in addition being covered with at least one of said materials.

Preferably the indicator is a self-contained biological indicator (SCBI) or a biological indicator strip, whereby a carrier of spores is made of paper, metals, glass, glass fiber, plastic, or any combination of these materials.

Preferably the indicator is a chemical indicator using various carriers like paper, glass fiber, stainless steel or plastic foil like PET, PP or others and is surface-protected or covered on both sides with different chemical indicator colours to be used to monitor different sterilization processes.

Advantageously the indicator of the indicator system is designed to achieve an especially high sensitivity. The detector volume is thereby primarily very low and extensively adapted to the volume that is taken by the actual indicator. Advantageously the detector volume is chosen smaller than about 250-500 pi, so that at the use of a common chemical or biological indicator with paper as carrier, consuming a volume of 100-250 ml, nearly half of the detector volume is filled up with the actual indicator.

As detector a system for the evaluation of physical parameters can be used, like for example a sensor for moisture, temperature, pressure and/or an ultrasonic sensor, which is located in a sterilization chamber, and may be designed also as a so-called data logger for wireless transmission of the received data. Solid materials like for example salts can be used as well, which change physically when the sterilization agent is present, so for example achieve their melting point and/or change their colour. Advantageous a chemical indicator is used as indicator, which changes its colour when in contact with the used sterilization agent, or a biological indicator, for example in the form of indicator strips and/or self-developed indica-tors. The test device is especially suitable monitoring sterilization processes with gaseous sterilization agents like for example low-temperature steam-, formaldehyde-, ethylene oxide-, hydrogen peroxide- or ozone-sterilization-processes.

The series gas-connection of several stages of the gas collection volume before the detector achieves specific current properties and the presence of a condensation area makes the test device also notably suitable, to allow specific condensation of the sterilization agent for the use in a sterilization process in which steam is used as the sterilization agent. Advantageous the process challenge test device is therefore used to monitor steam sterilization processes. Basically, all air removal versions for the sterilization chamber may be used. The test device in the process informs the operator, which steam penetration characteristics the process pro-vides.

The object of the invention is further achieved by a process challenge device, combination comprising as components two or more insertion parts and two or more housing parts, whereby these components are built in such a way that each insertion part can be combined with each housing part for providing a process challenge device as described above, whereby the insertion parts and/or housing parts are built differently such that each combination of insertion part and housing part differs from each other combination in its air removal and sterilant penetration characteristics.

A process challenge device combination or system allows by selecting one of the insertion components and one of the housing components to provide a process challenge device with certain properties/sensitivity which is needed or desired for the testing of the concrete process. In a very convenient way, various conditions can be tested by the selection of a suitable insertion piece and housing piece. Different configurations can simulate various difficulties of a successful sterilization.

Preferably the number of insertion parts and housing parts is identical.

Preferably, the process challenge device combination comprises three insertion parts and three housing parts. In this way, nine process challenge devices can be assembled which allow to simulate different sterilization conditions of instruments.

Preferably, the insertion parts have mutually different lengths of an outer hull which in the assembled state provides a wall of a gas volume and/or whereby said housing parts have mutually different lengths of an inner hull which in the assembled state provides a wall of a gas volume.

The invention also relates to a biological, chemical, or physical Indicator which is configured to be inserted into a process challenge device described above.

The advantages of the invention are especially as follows. Due to the two-part design of the process challenge device described above, the detector volume can be placed in the part to be inserted into the housing part. In this way, sterilizing agent and gas can only reach the detector volume by travelling through the whole series of volumes. Since the only way that sterilizing agent can reach the detector volume is by passing though all stages of the gas collection volume, a special and separate sealing of the detector volume is not necessary, increasing the lifetime and decreasing its maintenance intervals.

Due to the nested arrangement of the subsequent stages of the gas collection volume, the net volume of the process challenge device is reduced compared to traditional designs. The two-part design allows varying the various volumes/dimensions of the different gas compartments, thereby allowing adjusting of the sensitivity to individual sensitivity requirements.

With adjustment means and/or means for connecting both parts, no screwing connection is necessary, and the indicator/detector can be removed/inserted very quickly. Since both parts can be manufactured from long-term durable materials and no sealing is necessary, the process challenge device can be used for routine monitoring several thousand sterilization cycles. By providing the possibility to ad-just the extraction length of the insertion part with respect to the housing part, especially by clicking the insertion part to different positions, the process challenge device can be adjusted to special application requirements.

### BRIEF DESCRIPTION OF THE DRAWINGS

Further features and advantages of the present invention shall become clearer from the following detailed description of some of its preferred embodiments, made with reference to the attached schematic drawings and given as an indication and not for limiting purposes.

In particular, the attached drawings are included to provide a further understanding of the invention and are incorporated in and constitute a part of this specification. The drawings together with the description explain the principles of the invention.

In the drawings, corresponding characteristics and/or components are identified by the same reference numbers. In these drawings:

FIG. 1 shows a process challenge device with a housing part and an insertion part in a preferred embodiment in a perspective view;

FIG. 2 shows the housing part in a perspective way;

FIG. 3 shows a cross section through the housing part and the insertion part in an assembled state;

FIG. 4 shows the insertion part in a perspective way;

FIG. 5 shows a cross section through the housing part and the insertion part in an assembled state;

FIG. 6 shows a cross-sectional view with the insertion part inserted into the housing part;

FIG. 7 shows another cross-sectional view of the both parts fitted together;

FIG. 8 shows a housing part of a process challenge device in a second preferred embodiment in a perspective view;

FIG. 9 shows a cross section through the housing part and the insertion part in an assembled state;

FIG. 10 shows an insertion part of process challenge device in a further preferred embodiment;

FIG. 11 shows a cross section through the housing part and the insertion part in an assembled state;

FIG. 12 shows the insertion part of FIG. 10 fully inserted into a housing part;

FIG. 13 shows a cross section of the configuration according to FIG. 12;

FIG. 14 shows the process challenge device according to FIG. 12 with the insertion part loosely inserted into housing part;

FIG. 15 shows a cross section through the configuration of FIG. 14;

FIG. 16 shows a detailed view of the insertion part with adjustment means;

FIG. 17 shows a configuration with the insertion part fully inserted into housing part;

FIG. 18 shows a first configuration with the insertion part in a first adjusted position;

FIG. 19 shows a second configuration with the insertion part in a second adjusted position;

FIG. 20 shows the configuration with an indicator inserted into the detector volume;

FIG. 21 shows another cross-sectional view of the device;

FIG. 22 shows a process challenge device in a third preferred embodiment in a perspective view;

FIG. 23 shows the device of FIG. 22 in a side view;

FIG. 24 shows a cross-sectional view through the device of FIG. 22;

FIG. 25 shows a housing part of the device of FIG. 22 in a perspective view;

FIG. 26 shows the housing part in a cross-sectional view;

FIG. 27 shows the insertion part of the device of FIG. 22 in a perspective view;

FIG. 28 shows the inner part in a cross-sectional view;

FIG. 29 shows a process challenge device in a first cross-sectional view;

FIG. 30 shows the process challenge device of FIG. 29 in a second cross-sectional view;

FIG. 31 shows the process challenge device of FIG. 29 in a third cross sectional view;

FIG. 32 shows three components of a process challenge device system components in a preferred embodiment;

FIG. 33 shows three further components of a process challenge device system;

FIG. 34 shows a housing part of the process challenge device according to FIG. 29;

FIG. 35 shows the component of FIG. 34 in a sectional view;

FIG. 36 shows the component of FIG. 34 in a further sectional view;

FIG. 37 shows an insertion part of the process challenge device according to

FIG. 29 in a first view;

FIG. 38 shows the component of FIG. 37 in a further view;

FIG. 39 shows the component of FIG. 37 in a first sectional view; and

FIG. 40 shows the component of FIG. 37 in a second sectional view.

### DETAILED DESCRIPTION

In FIG. 1, a process challenge device 2 in a preferred embodiment is shown. The process challenge device 2 displayed in FIG. 1 shows a construction scheme de-signed to test the penetration characteristics of a sterilization agent. The knowledge achieved during the use of the test device 2 may be used especially for the verification or testing steam in a sterilization process. Using such a steam sterilization process the instruments or materials to be sterilized are put into a sterilization chamber not specified. At first the sterilization chamber is removed from air. The air removal process can be carried out by a downward gravity displacement process, super- or sub-atmospheric air removal cycles or their combinations. The process challenge device 2 is especially designed to test sterilizing properties of a sterilizing routine. The process challenge device 2 comprises a first or housing part 6 which provides an outer housing or casing 10. Partially and/or fully insertable into the first part 6 is a second or insertion part 14 which comprises a cover 18 for the casing 10.

Casing 10 has an oval shape which leads a reduced height needed when the process challenge device 2 is arranged in a sterilization chamber. In casing 10, an opening 22 is provided which is an entrance region for a multi-staged gas volume/series of gas volumes in which gas is transported in two opposing directions during the sterilization process and which are arranged in a spatially nested way inside casing 10. Casing 10 preferably comprises a part 26 with a recessed region 30. Casing 10 is closed at a first end 34 and provides an opening 36 at a second end 38 at which cover 18 is arranged on casing 10.

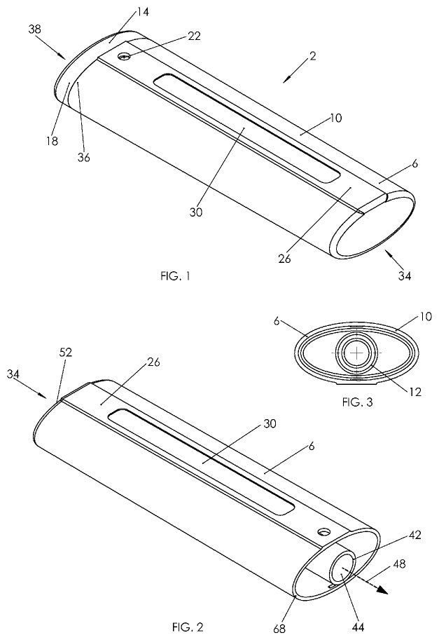

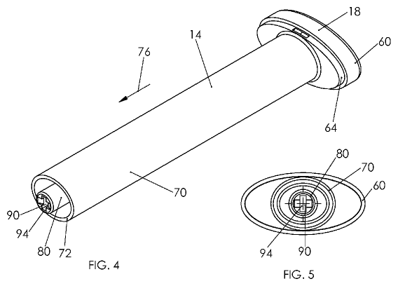

FIG. 2 shows the housing part 6 with the insertion part 14 removed. Housing part 6 comprises a second duct 42 or shell/hull which is shaped concentric to a common middle axis 48 of duct 42 and casing 10 and comprises an opening 44. Duct 42 is cylindrically shaped and is fixed to a bottom 52 from the inside of casing 10. An interior space of duct 42 is closed at the first end 34. A cross section through housing part 6 and insertion part 14 is shown in FIG. 3.

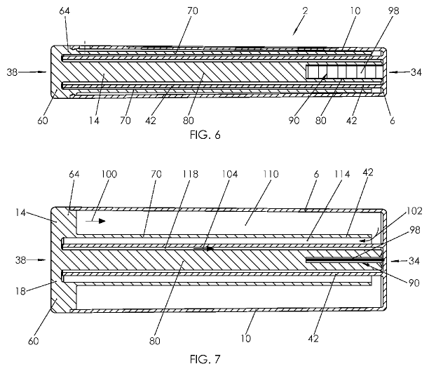

In FIG. 4, insertion part 14 which in the mounted position is at least partially insert-ed in housing part 6 is shown in a perspective view. Cover 18 is built as an oval-shaped piece with an end part 60 and an insertion part 64. In a fully inserted state, insertion part 64 is fully inserted in housing 10. In the fully inserted state, a rim 68 (see FIG. 2) of casing 10 serves as a seat for end part 60, which in lateral direction has a larger extension than insertion part 64. Second part 14 comprises a first duct 70 with an oval shape 72. First duct 70 in a mounted position extends into casing 10 in an axial direction 76 and leaves a passage region in the inside of casing 10 at first end 34. Inside first duct 70, a third duct 80 is arranged which has an outer circular shape. In axial direction 76, third duct 80 extends farther than first duct 70. A detector volume 90 with a detector holder 94 is arranged at the part of third duct 80 which preferably projects beyond first duct 70. A top view of second part 14 is shown in FIG. 5.

In a preferred method of manufacturing a process challenge device 2, the duct 42 is a part separate and separately manufactured from the casing 10. It preferably is cylinder-shaped piece which is put on a corresponding socket in the inside of casing 10. Third duct 80 of insertion part 14 is preferably a part separate and separately manufactured from duct 70. It is preferably inserted into duct 70 and put on a corresponding socket inside of duct 70.

The detector volume 90 is arranged at a dead end and closed end of casing 10 and is built in a sealing-less way, i.e. it does not comprise any sealing element and/or gasket. This has the advantage that no sealing element is necessary which in known devices needs regular checks and maintenance since a gas leak would render the measurement invalid. A sealing element is typically needed for an opening of the detector chamber for inserting and removing the detector. In the process challenge device 2 shown here, the detector volume 90 is arranged at an end of third duct 80 which is part of second or insertion part 14. When second or insertion part 14 is fully extracted from the first or housing part 6, the indicator/detector can be inserted into the detector volume 90. A separate sealed access to the detector volume 90 is not needed. Moreover, the detector volume 90 is only accessible by gas which has traversed the series of gas volumes provided in casing 10. The design with two parts 6, 14 and the location of detector volume 90 at an end part of second part 14 allows the reliable and sealing-less design shown.

In FIGS. 6 and 7, two sections through process challenge device 2 are shown in perpendicular orientations of device 2. When the process challenge device 2 is placed in a sterilization chamber, sterilizing agent and/or gas/steam can enter through the opening 22 and flow/move/stream in a direction 100 between first duct 70 and casing 10. More precisely, it will flow between an inner wall of casing 20 and an outer wall of first duct 70 by which a first gas volume 110 is defined. It will then continue to flow inside casing 10 to the first end 34. It will then flow in a direction 102 and continue its way in direction 102 between first duct 70 and second duct 42, i.e. between an inner wall or surface of first duct 70 and an outer wall of second duct 42 and continue in a second gas volume 114. It will then turn around and continue to flow in a direction 104 between first duct 42 and third duct 80 in a third gas volume 118 until it reaches the detector volume 90. In this way, a zig-zag path or passage is defined for the sterilizing agent. The described travel for gas

describes its way from opening 22 to detector volume 90. In a sterilization process, gas will move in both directions in gas volumes 110, 114, and 118.

The first passageway or volume 110 or first stage of the gas collection volume in the preferred embodiment has a volume of ca. 50 cm3. The second passageway or volume 114 or second stage of the gas collection volume in the preferred embodiment has a volume of ca. 15 cm³. The third passageway or volume 118 or third stage of the gas collection volume in the preferred embodiment has a volume of ca. 7 cm³.

Only if the sterilizing agent has passed through the whole passage from the entrance of the gas collection volume to the detector volume 90, an indicator/detector 98 placed in detector volume 90 will react to the sterilizing agent. Since the detector volume 90 is only accessible in this way, a high reliability of result if provided. The detector volume 90 does not have to be sealed in order to prevent direct access from sterilizing agent as it is the case in other test devices where the detector chamber has to be opened to insert the detector and has to be closed again, whereby a sealing element/gasket is provided for preventing direct entry of sterilizing agent.

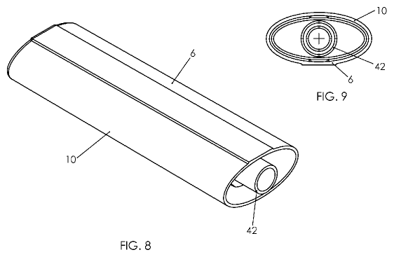

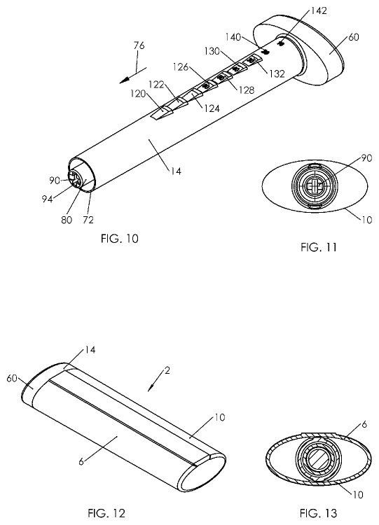

A housing part 6 of a process challenge device 2 in a second preferred embodiment is shown in FIG. 8. In contrast to the embodiment shown in the previous FIGs, the embodiment according to FIG. 8 does not provide an opening 22 as an entrance for gas. An entrance for gas to enter the series of volumes is made possible in a circumferential gap between both parts 6 and 14. In FIG. 9, first part 6 and second part 14 are shown in a cross section. As shown in FIG. 10, insertion part 14 in axial direction 76 comprises on duct 72 a series of engagement/adjustment elements 120, 122, 124, 126, 128, 130, 132. Adjustment elements 120-132 are preferably arranged equally spaced from each other and preferably comprise respective labels. The respective label preferably indicates a distance by which second part 14 is extracted from second part 14 compared to a fully inserted state.

Preferably, additional labels 140, 142 are provided which indicate extraction distances. In the present preferred embodiment, labels 140 and 142 indicate the numbers “20” and “10”, respectively, which denote an extraction distance of 20 or 10 millimeters. Adjustment elements 120, 124, 126, 128, 130, 132 are provided with the labels “60”,“50”,“40”,“30”, which again indicate extraction distances in millimeters. Adjustment elements are in the present embodiment build as ramps or teeth which protrude from duct 72 with increasing distance.

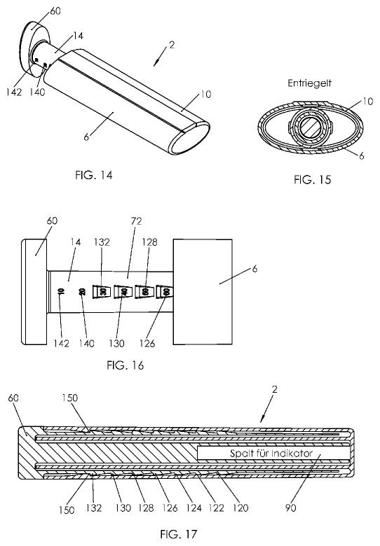

In FIG. 12, insertion part 14 is fully inserted into housing part 6. FIGS. 11 and 13 show cross sections through the parts 6, 14 fitted into each other.

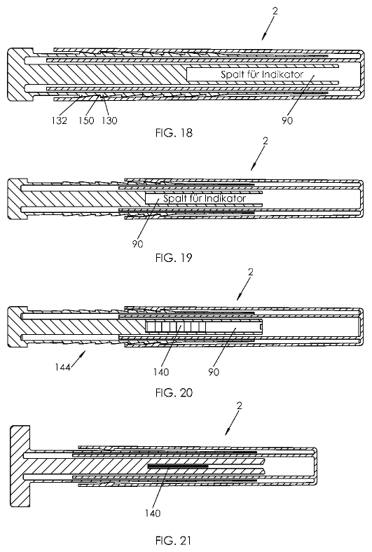

FIG. 14 displays insertion part 14 adjusted at a specified extraction length in housing part 6. In FIG. 14, end part 60 is tilted by 90 degrees compared to a configuration in which part 14 is engaged with housing part 6 at a specified extraction distance. Preferably, on opposite sides of insertion part 14, adjustment elements 126-132 and labels 140, 142 are provided. In this way, the engagement of insertion part 14 with housing part 6 is symmetric, leading to a better distribution of forces. Additionally, the handling for the user is improved. FIG. 15 shows a cross section of both components and FIG. 16 shows a detailed view of end part 60,

As can be seen in FIG. 17, on its inner side facing insertion part 14 in the mounted position, housing part 6 comprises protrusions 150 which can engage with adjustment elements 120-132. Preferably, protrusions 150 are arranged on opposite sides of the inner surface of first element 6. Protrusions 150 only cover a semi-circle of the inner circumference of element 6. In this way, when turning the insertion part 14 by a specified angle, protrusions 150 and adjustment elements 120-132 are disengaged.

In this configuration, insertion part 14 can be freely removed from housing part 6, for example for removing the present indicator and/or inserting a new indicator into detector volume 90 which is essentially built as a slot. Also, in the disengaged position, the extraction length of insertion part 14 with respect to housing part 6 can be chosen. Once the desired extraction length has been chosen and which can be inferred from the corresponding label, insertion part 14 can be turned for an engagement of the corresponding adjustment elements 120-132 with the protrusions 150. In the preferred embodiment, the fully engaged position of insertion element 14 in housing element 6 and the fully disengaged position are reached by a 90-degree rotation of insertion element 14 within housing element 6.

In FIG. 18, a configuration of insertion part 14 and housing part 6 is shown in which adjustment element 132 is engaged with protrusion 150 in a force-fitting manner. Due to the adjacent adjustment element 130, a movement of insertion part 14 to larger extraction lengths is blocked by protrusion 150.

FIGS. 19 and 20 show the process challenge device 2 in a second engaged position. In FIG. 20, the inserted indicator 140 is shown. Process challenge device 2 and indicator 144 build an indicator system 144. In FIG. 21, a view of the process challenge device rotated by 90 degrees is shown. With each additional distance by which the insertion part 14 is extracted from the housing part 6, the length of all three volumes which are arranged in series increases.

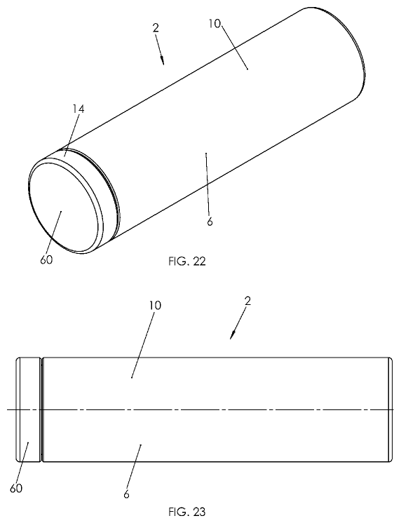

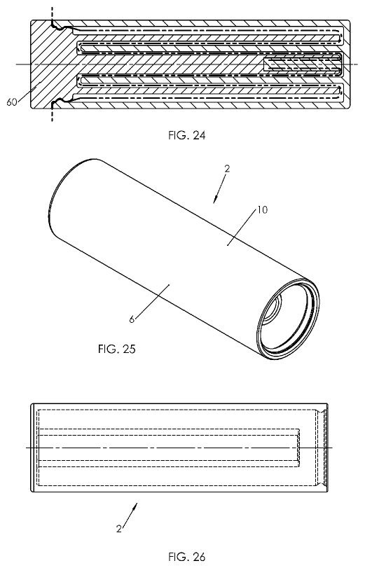

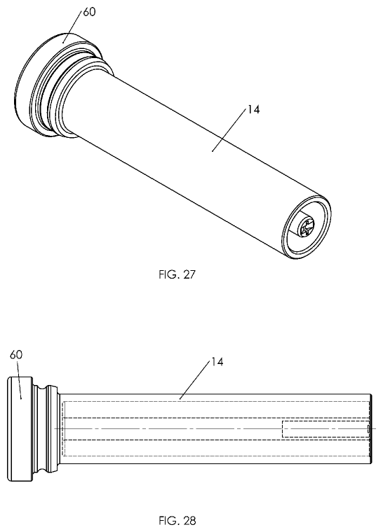

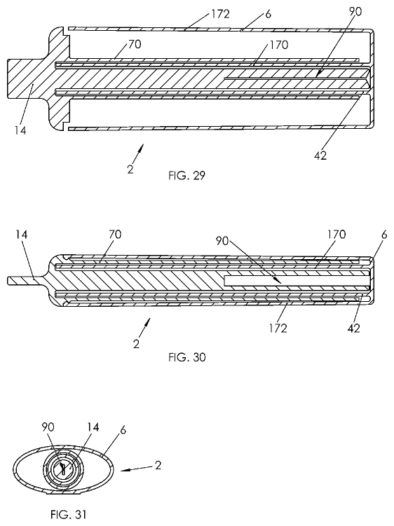

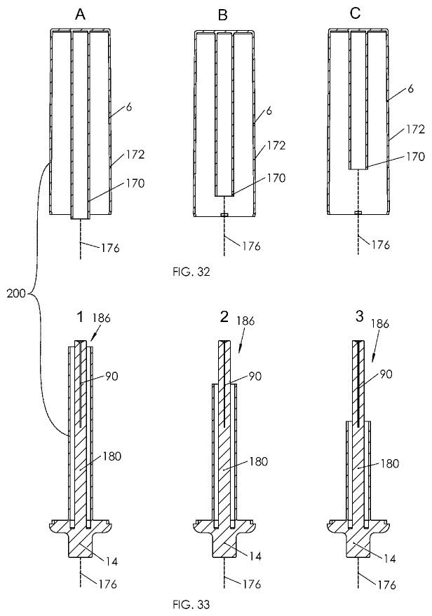

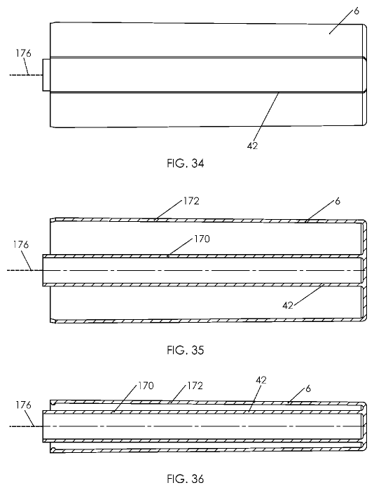

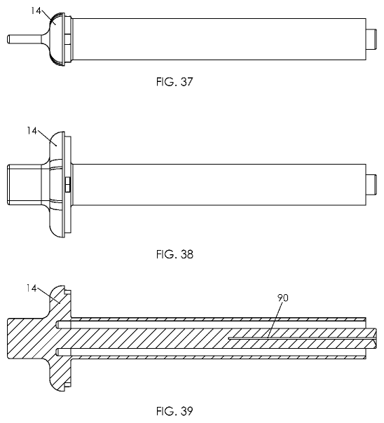

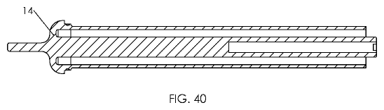

In FIGS. 22-28, a process challenge device 2 in further preferred embodiment is shown. In this embodiment, the housing part 6 has circular cross section. Preferably, also this embodiment is built with two parts 6, 14 as described in relation to the previous embodiment and provides a series of three gas volumes. Also, preferably, the design of the two parts 6, 14 in relation to casing and ducts is built as describe above.

When the insertion part 14 is only partially inserted into housing part 6, the entrance region for gas/steam is provided via a circular gap between end part 60/cover 18 and casing 10. The process challenge device can provide aligning means which allow entering the insertion part 14 into the housing part 6 only in a number of set of directions. If insertion part 14 and housing part 6 are then rotated with respect to each other by a certain angle, the insertion part 14 cannot be extracted from housing part 6. In this way, it can be reliably assured that the insertion part 14 is not released from housing part 6 during the sterilization process. In a preferred variant, in the inside of casing 10/housing part 6, at least one protrusion is arranged and whereby insertion part 14 comprises a collar with at least one recess which allows the passing of the protrusion when the insertion part 14 is inserted into housing part 6.

The collar is then arranged in a longitudinal direction arranged below said protrusion. When insertion part 14 is turned by a defined angle, the protrusion blocks the collar, thereby preventing in a form-locking manner the release of insertion part 14. Preferably, three protrusions are arranged equally spaced along an inner circumference and the collar comprises three recesses.

FIGS. 29-31 and 37 to 40 show a process challenge device in a further preferred embodiment. In this embodiment, an inner hull 170 of housing part 6 in an axial direction 176 extends further than an outer hull 172 of casing 6. In this way, the size of at least one gas volume formed between ducts/walls of parts 6/14 is altered in volume compared to the case in which both hulls 170, 172 have the same axial extension.

In FIG. 32 three different housing elements or housing parts 6 are shown. These housing parts 6 all comprise an identical outer hull 172. They also comprise, respectively, an inner hull 170 which in an assembled state with three insertion parts 14 shown in FIG. 33 form ducts or gas passages for gas which enters the process challenge device. The housing parts 6 shown in FIG. 32 differ in the length of inner duct or hull 170 in an axial direction 176. The housing part 6 shown on the left comprises the longest, the housing part 6 shown on the right comprises the shortest hull 170.

The three insertion parts 14 shown in FIG. 33 differ by the extension in axial direction 176 of an inner part 180 in which at one end 186 the detection volume 90 is formed.

Housing parts 6 according to FIG. 32 and insertion parts 14 according to FIG. 33 constitute a process challenge device system 200. Each insertion part shown in FIG. 33 can be inserted in each housing part 6. In this way, nine different process challenge devices 2 can be built which differ from each other in at least one dimension, especially length and/or cross section of a gas volume. This set of process challenge devices therefore allows simulating different sterilization sensitivities. This modular system saves material needed for components. If nine process challenge devices would have to be built separately, nine instead of three housing and insertion parts would be necessary. This system can also be extended easily by adding one or more housing parts 6 and/or insertion parts 14.

1. Process challenge device (2), especially for simulating the worst-case penetration conditions of a load inside a sterilization chamber, comprising a detector volume (90) for housing a biological, chemical or physical indicator (140), whereby said detector volume (90) is arranged at a dead end of a series of gas volumes (110, 114, 118), characterized in that said process challenge device (2) comprises two parts (6, 14), namely a housing part (6) and an insertion part (14), whereby said insertion part (14) is at least partially insertable into said housing part (6) in such a way that said detector volume (90) is arranged without any seal inside said both parts (6, 14).

2. Process challenge device (2) according to claim 1 , whereby said two parts (6, 14) fit into each other without sealing and form, combined together, in series connected volumes (110, 114, 118) for providing specific penetration characteristics for sterilization agents where the gas entrance inlet/outlet is between the connection of the housing and insertion part.

3. Process challenge device (2) according to claim 1, whereby said series of volumes (110, 114, 118) provides connected channels, whereby one end of said channels is connectable to a sterilization chamber and the other end is connected to said detector volume (90) which is configured to house, alternatively one or more chemical, biological, or physical indicators for detecting the presence of a sterilizing agent.

4. Process challenge device (2) according to claim 1, whereby said two parts (6, 14) comprise means (120-132; 150) for a force-fit and/or form-fit connection, especially by clicking or snapping together by mechanically fixing their positions.

5. Process challenge device (2) according to claim 4, whereby adjusting means (120-132) are provided for positioning said two parts (6, 14) with respect to each other in a plurality of defined positions with respect to each other by mechanically fixing the position.

6. Process challenge device (2) according to claim 1, whereby said housing (6) and/or said insertion part (14) are made of metal, plastic, or metal-plastic-bonded system.

7. Process challenge device (2) according to claim 1, whereby from an entrance region to said dead end said in series connected gas volumes (110, 114, 118) decrease in volume and/or cross section.

8. Process challenge device (2) according to claim 1, whereby at least one of said parts (6, 14) comprises a round, oval, rectangular, or multi-edge shape.

9. Process challenge device (2) according to claim 1, whereby said series of gas volumes (110, 114, 118) are partially or totally filled with porous material.

10. Process challenge device (2) according to claim 1, whereby said detector volume (90) has a volume of less than 200 microliters, a length between 3 and 6 cm and a cross section between 3 and 5 mm2.

11. Indicator system (144), comprising a process challenge device (2) according to one of the previous claims and at least one chemical, biological, or physical indicator (140) arranged in said detector volume (90).

12. Indicator system (144) according to claim 11, with a biological indicator (140), whereby a carrier of said biological indicator (98, 140) is made from paper, metal, glass fiber, plastic, stainless steel, any plastic foil, Tyvek or any combi-nation, especially in addition being covered with at least one of said materials.

13. Indicator system (144) according claim 11, whereby said indicator (98, 140) is a self-contained biological indicator (SCBI) or a biological indicator strip, whereby a carrier of spores is made of paper, metals, glass, glass fiber, plastic or any combination of said materials.

14. Indicator system (144) according to claim 12, whereby the indicator (98, 140) is a chemical indicator using various carriers like paper, glass fiber, stainless steel or plastic foil like PET, PP or others and is surface-protected or covered on both sides with different chemical indicator colours to be used to monitor different sterilization processes.

15. Process challenge device combination (200), comprising as components two or more insertion parts (14), and two or more housing parts (6), whereby said components are built in such a way that each insertion part (14) can be combined with each housing part (6) for providing a process challenge device (2) according to claim 1, whereby said insertion parts (14) and/or housing parts (6) are built differently such that each combination of insertion part (14) and housing part (6) differs from each other combination in its air removal and sterilant penetration characteristics, which preferably allows to make several different PCD with only a few housing and insertion parts.
